SEQUENCE LISTING (2 sequences)

SEQ ID NO: 1 - DNA, 34 bp, Artificial Sequence
  Synthetic polynucleotide
tacactctttccctacacgacgctcttccgatct

SEQ ID NO: 2 - DNA, 34 bp, Artificial Sequence
  Synthetic polynucleotide
gtgactggagttcagacgtgtgctcttccgatct

(19) United States
(12) Patent Application Publication
(10) Pub. No.: US 2023/0093891 A1
(43) Pub. Date: Mar. 30, 2023

(54) MICROFLUIDIC CELL BARCODING AND SEQUENCING

(71) Applicants: CHAN ZUCKERBERG BIOHUB, INC., San Francisco, CA (US); THE REGENTS OF THE UNIVERSITY OF CALIFORNIA, Oakland, CA (US)

(72) Inventors: Aaron Streets, Oakland, CA (US); Tyler Chen, Oakland, CA (US); Anushka Gupta, Oakland, CA (US)

(21) Appl. No.: 17/798,501

(22) PCT Filed: Feb. 12, 2021
(86) PCT No.: PCT/US21/17804

§ 371 Date: Aug. 9, 2022

Related U.S. Application Data
(60) Provisional application No. 62/976,446, filed on Feb. 14, 2020.

(51) Int. Cl. C12Q 1/6869; C12Q 1/6806

(52) U.S. Cl. CPC C12Q 1/6869; C12Q 1/6806

(57) ABSTRACT

The present disclosure provides materials and methods to link imaging and sequencing measurements of a single cell. Sequencing information can be linked with phenotypic measurements that are not directly encoded in the genome such as morphological features, protein expression & localization, organelle dynamics, or the metabolic composition of a cell.

### STATEMENT REGARDING FEDERALLY SPONSORED RESEARCH

The invention was made with Government support under Grant No. GM124916 awarded by NIH National Institute of General Medical Sciences. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “54664_Seqlisting.txt”, which was created on Feb. 11, 2021 and is 656 bytes in size. The subject matter of the Sequence Listing is incorporated herein in its entirety by reference.

### FIELD

The present disclosure relates generally to methods for linking imaging and sequencing measurements of single cells.

### BACKGROUND

Over the last decade, single-cell genomics has revolutionized the study of complex biological systems, enabling the characterization of cell-to-cell heterogeneity that underlies all bulk properties at the systems level. Most notably, single-cell RNA-sequencing (scRNA-seq), which involves reverse transcription of mRNA followed by high-throughput sequencing of cDNA, has allowed profiling of the whole transcriptome of individual cells in an unbiased manner. Implementation of scRNA-seq requires the isolation of individual cells, which makes it challenging and costly to increase throughput with standard microliter-scale plate-based protocols. Recently, these limitations on scalability have been overcome by the development of microwell- and microdroplet-based approaches for scRNA-seq (G. X. Y. Zheng, et al., Nat. Commun., DOI:10.1038/ncomms14049; E. Z. Macosko, et al., Cell, 2015, 161, 1202-1214; A. M. Klein, et al., Cell, 2015, 161, 1187-1201; J. Yuan and P. A. Sims, Sci. Rep., DOI:10.1038/srep33883; and T. M. Gierahn, et al., Nat. Methods, 2017, 14, 395-398). These methods use microfabricated devices to isolate cells in nanoliter volumes, in which cellular barcodes (T. Hashimshony, et al., 2012, 2, 666-673) and unique molecular identifiers (UMIs) (S. Islam, et al., Nat. Methods, 2014, 11, 163-166) are incorporated into cDNA by reverse transcription. This allows for multiplexed, parallel processing of many cells with absolute transcript quantification by UMI counting. The rapid development of these high throughput scRNA-seq protocols (C. Ziegenhain, et al., Mol. Cell, 2017, 65, 631-643.e4.; V. Svensson, et al., Nat. Methods, 2017, 14, 381-387; and J. Ding, et al., bioRxiv, 2019, 632216) and the simultaneous expansion of bioinformatics tools have now made it possible to analyze hundreds to thousands of single cells in one experiment, thereby enabling researchers to construct transcriptional atlases at an organ- (S. Darmanis, et al., Proc. Natl. Acad. Sci. U. S. A., 2015, 112, 7285-7290; A. Zeisel, et al., Science (80-.)., 2015, 347, 1138-1142; M. J. Muraro, et al., Cell Syst., 2016, 3, 385-394.e3; and E. M. Kernfeld, et al., Immunity, 2018, 48, 1258-1270.e6) and organism-level (T. T. M. Consortium, S. R. Quake, T. Wyss-Coray and S. Darmanis, bioRxiv, 2017, 237446; and X. Han, et al., Cell, 2018, 172, 1091-1107.e17).

While scRNA-seq allows researchers to genotype a large number of cells, it lacks the ability to detect phenotypic measurements that are not directly encoded in the genome such as morphological features, protein expression & localization, organelle dynamics, or the metabolic composition of a cell (A. Gupta, et al., Analyst, 2019, 144, 753-765). A multitude of fluorescence-based and label-free imaging modalities have long been used to acquire such phenotypic data from live cells, and since cells remain intact during image-based measurements, scRNA-seq can be performed directly after microscopy. This way, imaging and sequencing measurements can be made on the same single cell. Examination of such linked measurements using dedicated analysis tools for multi-omic data would enable researchers to start understanding the transcriptional underpinnings of observed cellular attributes.

While microdroplet- and microwell-based barcoding have significantly increased the throughput of scRNA-seq, these protocols lack the ability to link imaging and sequencing measurements due to the random pairing between a cell and its DNA barcode. Recently, Lane et al. reported a perturbation assay using epifluorescence microscopy linked with scRNA-seq on the Fluidigm C1 microfluidic platform using a non-barcoding based library preparation protocol (K. Lane, et al., Cell Syst., 2017, 4, 458-469). However, C1-based methods have limited scalability since each individual cell requires library preparation in-tube. In another promising demonstration, Yuan et al. engineered optically decodable beads for combining imaging and sequencing in a scalable fashion. Throughput comes at the cost of reduced phenotypic information, however, making this assay most useful for low-resolution widefield imaging of multiple cells at a time (J. Yuan, J. Sheng and P. A. Sims, Genome Biol., 2018, 19, 227).

Thus there remains a need in the art to link imaging and sequencing measurements of a single cell.

### SUMMARY OF THE INVENTION

The present disclosure provides, in various aspects, methods and materials to link imaging and sequencing measurements of a single cell. As provided herein, sequencing information, including the genotype one or more single cells, can be linked with phenotypic measurements that are not directly encoded in the genome such as morphological features, protein expression & localization, organelle dynamics, or the metabolic composition of a cell.

One aspect of the present disclosure provides a method of determining the sequence of one or more transcribed genes from a single cell, said method comprising the steps of: (a) administering a collection of cells to one lane of a microfluidic device under conditions that allow a single cell from the collection of cells to enter a first chamber in the microfluidic device; (b) capturing a single cell in a trapping chamber of the microfluidic device; (c) flowing the single cell to a lysis chamber pre-loaded with barcoded reverse-transcription primers; (d) preparing a barcoded cDNA library from the single cell using the barcoded reverse-transcription primers under conditions that allow barcoded cDNA preparation; (e) sequencing the barcoded cDNA; wherein steps (b)-(d) are carried out in in the microfluidic device.

In a related aspect, the aforementioned method further comprises determining the abundance of the one or more transcribed genes.

In another aspect, the aforementioned method is provided wherein step (b) additionally comprises the step of collecting a non-invasive measurement of the single cell.

In still another aspect, the non-invasive measurement comprises an optical measurement. In various aspects, the optical measurement is selected from the group consisting of spectroscopy, light scattering imaging, and fluorescent lifetime imaging. In one aspect of the present disclosure, the optical measurement comprises capturing an image of the single cell. In yet other various aspects, the image of the cell is captured from a device selected from the group consisting of a camera, a microscope, an inverted microscope, a wide-field fluorescent microscope, a scanning confocal microscope, a nonlinear optical microscope, a two-photon fluorescent microscope, and a coherent Raman microscope.

In yet another aspect, an aforementioned method is provided which additionally comprises the step of linking the sequence obtained in step (e) with the image captured in step (b), thereby correlating expression of one or more transcribed genes to a single cell morphology or phenotype.

In another aspect, an aforementioned method is provided wherein the preparing of barcoded cDNA of step (d) comprises the steps of: (i) lysing the cell, (ii) re-suspending the barcoded primers, (iii) administering reagents and applying temperatures that allow cDNA preparation, and (iv) collecting the barcoded cDNA library. In one aspect, the lysing step comprises contacting the cell with a cell lysing agent selected from the group consisting of ionic and non-ionic detergents, Triton X-100, sodium dodecyl sulfate (SDS), NP-40, and ammonium chloride potassium.

In still another aspect, an aforementioned method is provided wherein the microfluidic device comprises 1-100 separate lanes, each comprising at least one chamber. In some aspects, each lane comprises 1, 2, 3, 4, 5, 6, 7, 8, 9, 10, 11, or 12 separate chambers. In some aspects, the cDNA from 1, 2, 3, 4, 5, 6, 7, 8, 9, or 10 lanes of the microfluidic device are pooled prior to sequencing.

In another aspect, an aforementioned method is provided wherein the transcribed gene is selected from the group consisting of a chromosomal-derived gene and a plasmid-derived gene. In another aspect, an aforementioned method is provided wherein the cell is a bacterial cell, a eukaryotic cell or prokaryotic cell. In one aspect, the cell is a mammalian cell. In another aspect, the cell is a human cell.

In one aspect, the present disclosure provides a method of determining the sequence of one or more transcribed genes from a single cell, said method comprising the steps of: (a) administering a collection of cells to one lane of a microfluidic device under conditions that allow a single cell from the collection of cells to enter a first chamber in the microfluidic device; (b) capturing a single cell in a trapping chamber and collecting an image of the single cell; (c) flowing the singe cell to a lysis chamber pre-loaded with barcoded reverse-transcription primers; (d) preparing a barcoded cDNA library from the single cell using the barcoded reverse-transcription primers under conditions that allow barcoded cDNA preparation; (e) sequencing the barcoded cDNA; wherein steps (b)-(d) are carried out in in the microfluidic device.

### BRIEF DESCRIPTION OF THE DRAWINGS

FIGS. 1A-1E show a μCB-seq device design and workflow. FIG. 1A: Schematic of the microfluidic device with control layer in dark gray and flow layer in light gray. For chip operation, cells are loaded into the cell inlet, reagent is introduced through reagent inlet and waste is collected via waste output. The device processes 10 cells, each in its individual reaction lane that ends in an output port. Reverse-transcribed cDNA is recovered from output ports for all cells. FIG. 1B: Detailed diagram of one reaction lane showing imaging, lysis and RT module. The imaging module has one imaging chamber, the lysis module has three reaction chambers and the RT module has one reaction chamber and a connective channel. Cell enters from the top and is isolated in the imaging chamber for imaging. Post-imaging, reagents are injected into the lane along with the cell for library preparation. This process is parallelized across all 10 lanes. Mixing paddles (MP1, MP2 and MP3) are actuated to homogenize reaction mixes during lysis and RT. Barcoded primers are added to the cDNA in the RT chamber. FIG. 1C: Detailed diagram of the imaging module showing the imaging chamber surrounded by two isolation valves that are actuated to actively capture cells of interest in the imaging chamber. FIG. 1D: Structure of the RT primer used in μCB-seq for pooling. The primers have 8-nt long known barcode sequence and 10-nt long unique molecular identifier (UMI) sequence. Primers are spotted in lysis chamber 3 and re-suspended in the lysis mix by actuating MP3. FIG. 1E: cDNA sequencing experimental pipeline. cDNA recovered from all 10 output ports is pooled in one tube for off-chip library preparation using mcSCRB-seq protocol and sequenced using next-generation sequencing platforms.

FIGS. 2A-2D shows the validation of primer recovery from PDMS spotting. Fragment analysis size distribution traces for barcoded primers that were suspended in nuclease-free water at RT FIG. 2A left in the original tube, FIG. 2B Spotted on PDMS, FIG. 2C baked at 80° C. and recovered by resuspending in nuclease-free water. FIG. 2D Qubit concentrations for condition B. The increase in concentration over baseline is likely due to evaporation of some small fraction of the 2 uL of water during the resuspension process.

FIGS. 3A-3E show the characterization of total RNA libraries generated using μCB-Seq. 20 libraries of 10 pg total RNA were sequenced using μCB-seq. FIG. 3A: Distribution of percent exonic, intronic, intergenic, ambiguous and unmapped reads in each of the 20 libraries. FIG. 3B: Number of genes detected (umi-count>0) in each of the 20 libraries sequenced to a depth of 30,000 reads per sample. FIG. 3C: Distribution of correlation in gene expression profile for all pairs possible amongst the 20 libraries (n=190 pairs). Pearson Correlation coefficients were calculated for genes detected in at least one of the 20 libraries. FIG. 3D: Genes detected in a pool of the 20 libraries sequenced to a depth of ˜1.3 million reads (grey circle) compared with the genes detected in a bulk library (TPM>0) prepared using 1000 ng total RNA and sequenced to the same depth (red circle). FIG. 3E: Scatter plot shows correlation in gene expression profile between the pool of 20 libraries and the bulk library prepared using 1000 ng total RNA. Correlation coefficient was calculated using genes detected in either bulk sample or one of the 20 total RNA libraries.

FIGS. 4A-4D show μCB-seq is more sensitive than in-tube mcSCRB-seq protocol. FIG. 4A: Median genes detected for downsampled read depth across single HEK cells sequenced using μCB-seq and mcSCRB-seq. μCB-seq detected significantly higher genes for read—depth>=40000 as tested by two-group Mann-Whitney U-test (p-value<.01). Error bars indicate the interquartile range. FIG. 4B: The ratio of genes detected (umi-count>0) in the single-cell libraries sequenced to an average depth of 200,000 reads to the genes detected in the bulk library (TPM>0) binned by expression level (bin width=0.1). Bulk library was prepared using 1000 ng total RNA and sequenced to a depth of 63 million reads. Error bars indicate interquartile range (n=16 cells each for both protocols). For bin width 3.6<log 10(TPM+1)<3.7 (marked by +), only one out of three genes was detected in all single cells across both protocols and was considered an outlier for loess regression. FIG. 4C: A zoomed-in plot of FIG. 4B comparing fraction of genes detected in the two protocol with low- and medium-abundance in bulk measurement (9<TPM<79). FIG. 4D: The coefficient of variation (SD normalized by the mean, n=16 cells in each protocol) is plotted against the bulk expression for genes commonly detected in bulk, μCB-seq and mcSCRB-seq. The highlighted region displays the 95% confidence interval around the smooth fit as determined by loess.

FIGS. 5A-5D show Linked imaging and sequencing using μCB-seq. FIG. 5A: Montage of representative images of HEKs and Preadipocytes acquired using scanning transmission and scanning confocal microscopy in the green and red channel. HEKs and Preadipocytes were stained with CellBrite green and red cytoplasmic membrane dye respectively. White arrowheads point towards subcellular features observed in images at a resolution of 209 nm per pixel. FIG. 5B: Normalized fluorescence signal in the green and red channel confocal images of both HEKs and Preadipocytes. Analysis of images for cell-mask generation and quantification of fluorescent intensities is explained in Methods section. FIG. 5C: Accurate identification of HEKs and Preadipocytes as two cell populations using unsupervised hierarchical clustering in the principal component space. Top 2000 most variable features were used as an input for determining the first two principal components. FIG. 5D: Unsupervised hierarchical clustering using scaled expression values of top-16 upregulated genes in HEKs and Preadipocytes. Heat map shows z-scored expression values for the 32 genes. On the bottom are heat map visualizations of normalized fluorescence intensities plotted in FIG. 5B. The heat maps for green and red channel are ordered to accurately reflect a one-on-one correspondence between imaging and sequencing data points.

FIGS. 6A-6E show the fabrication of μCB-seq devices with barcoded RT primer spotting. FIG. 6A: Photolithographic patterning of control and flow molds on Si wafers. FIG. 6B: Diagram of PDMS casting and alignment of the control and flow layers for undercured PDMS bonding between the two layers. FIG. 6C: Detailed diagram of barcoded RT primer spotting. Unique primers are delivered to each lysis module and dried before the device is closed. FIG. 6D: shows bonding of the primed device to a PDMS dummy layer to close the flow layers. FIG. 6E: PDMS devices are then plasma bonded to a coverglass for final assembly. The scale bar refers to FIGS. 6A to 6E.

### DETAILED DESCRIPTION

The present disclosure addresses the aforementioned need in the art and provides microfluidic cell barcoding and sequencing (μCB-seq) materials as well as microfluidic-based methods to extract both high-resolution optical imaging and highly sensitive scRNA-seq data from the same single cells in a multiplexed fashion. As provided below, the methods provide preloading addressable reaction chambers in our microfluidic device with known barcoded primers and re-suspending them with cell lysate during chip operation. Cells are individually trapped on the device using integrated on-chip valves and then imaged, upstream of library preparation. Since only one cell is imaged at a time, μCB-seq has the ability to characterize phenotypic information requiring high-resolution imaging or even time-resolved imaging to investigate dynamic cellular behavior. On-chip library preparation is carried out using a molecular crowding single-cell RNA barcoding and sequencing (mcSCRB-seq) protocol (W. Bagnoli, et al., Nat. Commun., DOI:10.1038/s41467-018-05347-6) which was shown to be the most sensitive protocol amongst contemporary scRNA-seq techniques when benchmarked using ERCC spike-ins. As described herein, μCB-seq improves upon the high sensitivity of mcSCRB-seq by utilizing the benefits of efficient, automated, and low-volume library preparation reactions at the microscale. Using a multiplexed scRNA-seq protocol also enables pooling libraries after reverse-transcription, making μCB-seq a scalable method for linking high information content optical and RNA-seq data from the same single cells.

### Definitions

The terms “polynucleotide” and “nucleic acid” refer to a polymer composed of a multiplicity of nucleotide units (ribonucleotide or deoxyribonucleotide or related structural variants) linked via phosphodiester bonds. A polynucleotide or nucleic acid can be of substantially any length, typically from about six (6) nucleotides to about 10⁹ nucleotides or larger. Polynucleotides and nucleic acids include RNA, cDNA, genomic DNA. In particular, the polynucleotides and nucleic acids of the present invention refer to polynucleotides encoding a chromatin protein, a nucleotide modifying enzyme and/or fusion polypeptides of a chromatin protein and a nucleotide modifying enzyme, including mRNAs, DNAs, cDNAs, genomic DNA, and polynucleotides encoding fragments, derivatives and analogs thereof. Useful fragments and derivatives include those based on all possible codon choices for the same amino acid, and codon choices based on conservative amino acid substitutions. Useful derivatives further include those having at least 50% or at least 70% polynucleotide sequence identity, and more preferably 80%, still more preferably 90% sequence identity, to a native chromatin binding protein or to a nucleotide modifying enzyme.

The term “oligonucleotide” refers to a polynucleotide of from about six (6) to about one hundred (100) nucleotides or more in length. Thus, oligonucleotides are a subset of polynucleotides. Oligonucleotides can be synthesized manually, or on an automated oligonucleotide synthesizer (for example, those manufactured by Applied BioSystems (Foster City, Calif.)) according to specifications provided by the manufacturer or they can be the result of restriction enzyme digestion and fractionation.

The term “primer” as used herein refers to a polynucleotide, typically an oligonucleotide, whether occurring naturally, as in an enzyme digest, or whether produced synthetically, which acts as a point of initiation of polynucleotide synthesis when used under conditions in which a primer extension product is synthesized. A primer can be single- stranded or double-stranded. As described herein, in some aspects of the present disclosure, the primer or primers are immobilized within or on a microfluidic device such as a device described herein.

The term “nucleic acid array” as used herein refers to a regular organization or grouping of nucleic acids of different sequences immobilized on a solid phase support at known locations. The nucleic acid can be an oligonucleotide, a polynucleotide, DNA, or RNA. The solid phase support can be silica, a polymeric material, glass, beads, chips, slides, or a membrane. The methods of the present invention are useful with both macro- and micro-arrays. In some embodiments, the nucleic acid array is immobilized within or on a microfluidic device such as a device described herein.

The term “protein” or “protein of interest” refers to a polymer of amino acid residues, wherein a protein may be a single molecule or may be a multi-molecular complex. The term, as used herein, can refer to a subunit in a multi-molecular complex, polypeptides, peptides, oligopeptides, of any size, structure, or function. It is generally understood that a peptide can be 2 to 100 amino acids in length, whereas a polypeptide can be more than 100 amino acids in length. A protein may also be a fragment of a naturally occurring protein or peptide. The term protein may also apply to amino acid polymers in which one or more amino acid residues is an artificial chemical analogue of a corresponding naturally occurring amino acid. A protein can be wild-type, recombinant, naturally occurring, or synthetic and may constitute all or part of a naturally-occurring, or non-naturally occurring polypeptide. The subunits and the protein of the protein complex can be the same or different. A protein can also be functional or non-functional.

The term “polypeptide” refers to a polymer of amino acids and its equivalent and does not refer to a specific length of the product; thus, peptides, oligopeptides and proteins are included within the definition of a polypeptide. A “fragment” refers to a portion of a polypeptide having typically at least 10 contiguous amino acids, more typically at least 20, still more typically at least 50 contiguous amino acids of the chromatin protein. A “derivative” is a polypeptide which is identical or shares a defined percent identity with the wild-type chromatin protein or nucleotide modification enzyme. The derivative can have conservative amino acid substitutions, as compared with another sequence. Derivatives further include, for example, glycosylations, acetylations, phosphorylations, and the like. Further included within the definition of “polypeptide” are, for example, polypeptides containing one or more analogs of an amino acid (e.g., unnatural amino acids, and the like), polypeptides with substituted linkages as well as other modifications known in the art, both naturally and non-naturally occurring. Ordinarily, such polypeptides will be at least about 50% identical to the native chromatin binding protein or nucleotide modification enzyme acid sequence, typically in excess of about 90%, and more typically at least about 95% identical. The polypeptide can also be substantially identical as long as the fragment, derivative or analog displays similar functional activity and specificity as the wild-type chromatin protein or nucleotide modification enzyme.

The terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence, as measured using one of the following sequence comparison algorithms, or by visual inspection.

The phrase “substantially identical,” in the context of two nucleic acids or polypeptides, refers to two or more sequences or subsequences that have at least 60%, typically 80%, most typically 90-95% nucleotide or amino acid residue identity, when compared and aligned for maximum correspondence, as measured using one of the following sequence comparison algorithms, or by visual inspection. An indication that two polypeptide sequences are “substantially identical” is that one polypeptide is immunologically reactive with antibodies raised against the second polypeptide.

“Similarity” or “percent similarity” in the context of two nucleic acids or polypeptides, refer to two or more sequences or subsequences that are the same or have a specified percentage of nucleotides or amino acid residues or conservative substitutions thereof, that are the same, when compared and aligned for maximum correspondence, as measured using one of the following sequence comparison algorithms, or by visual inspection. By way of example, a first sequence can be considered similar to a second sequence when the first sequence is at least 30%, 40%, 50%, 60%, 70%, 75%, 80%, 90%, or even 95% identical, or conservatively substituted, to the second sequence when compared to an equal number of nucleotides or amino acids as the number contained in the first sequence, or when compared to an alignment that has been aligned by a computer similarity program known in the art, as discussed below.

Generally, other nomenclature used herein and many of the laboratory procedures in cell culture, molecular genetics and nucleic acid chemistry and hybridization, which are described below, are those well-known and commonly employed in the art. (See generally Ausubel et al. (1996) supra; Sambrook et al, Molecular Cloning: A Laboratory Manual, Second Edition, Cold Spring Harbor Laboratory Press, New York (1989), which are incorporated by reference herein). Standard techniques are used for recombinant nucleic acid methods, polynucleotide synthesis, preparation of biological samples, preparation of cDNA fragments, isolation of mRNA and the like. Generally enzymatic reactions and purification steps are performed according to the manufacturers' specifications.

### Linking High-Resolution Optical Imaging and Highly Sensitive scRNA-Seq Data from the Same Single Cells in a Multiplexed Fashion

The present disclosure provides methods and materials for co-determining or linking imaging and sequencing measurements of a single cell.

Microfluidic technologies have been at the core of the recent exponential increase in throughput of scRNA-seq techniques, paving the way for undertakings such as the Human Cell Atlas Project (A. Regev, et al., Elife, DOI:10.7554/eLife.27041). However, scRNA-seq can only record information encoded as a sequence of nucleotides. Orthogonal measurements enabled by quantitative live-cell-imaging-based assays such as immunofluorescence (J. R. Lin, et al., Nat. Commun., DOI:10.1038/ncomms9390), subcellular lipid quantification (C. Cao, et al., Anal. Chem., 2016, 88, 4931-4939) or organelle-level pH measurements (H. Hou, et al., Sci. Rep., DOI:10.1038/s41598-017-01956-1) allow characterization of the phenotypes that also play a critical role in governing the functional state of a cell. Linking the two measurements as provided for the first time herein thereby allows the correlation between gene expression and cellular traits. In the present disclosure, μCB-seq provides a scalable microfluidic platform which allows acquisition of high-resolution images and RNA-sequencing libraries from the same single cells. As disclosed herein, μCB-seq devices are preloaded with known barcode sequences spotted at addressable locations, which allows linking these measurements.

As discussed herein, the preloaded (e.g., “imprinted”) barcodes can be recovered with high efficiency during chip operation even after being baked at 80° C. for 2 hours. The microfluidic device also features a modular design that allows for multistep scRNA-seq library preparation on-chip. While this uses a single barcoding step for scRNA-seq, it is contemplated that this on-chip barcoding approach is useful for many-step reactions in which aqueous samples can be automatically directed to multiple preloaded chambers for combinatorial spatial barcoding, targeted gene expression (H. C. Fan, et al., Science (80-.)., 2015, 347, 1258367), or CRISPR-based gene editing (H. Sinha, et al., Lab Chip, 2018, 18, 2300-2312).

As described herein, a method of determining the sequence of one or more transcribed genes from a single cell is provided. While the proof of principle in the present examples addresses RNAseq, it is contemplated the materials and methods provided herein can be be used to barcode any kind of genomic measurement including DNAseq, DamID seq, ATACseq, and others known in the art. The methods described herein allow determining the abundance of the one or more transcribed genes. In various aspects, the transcribed genes represents the transcriptome of the single cell.

In addition to sequence determination, the methods described herein provide the collection of a non-invasive measurement of the single cell. By way of non-limiting example, one aspect provides the capture or collection of an image of the single cell. Additional optical measurements are also contemplated by the present disclosure. Other kinds of measurements that can be coupled with uCB-SEQ include, but are not limited to, electrical measurements, physical measurements. In this way, uCB-SEQ enables any sort of non-invasive or non-perturbative measurement to be linked with any genomic measurement.

In various embodiments, optical measurements include spectroscopy, light scattering imaging, and fluorescent lifetime imaging. In various embodiments, the optical image is captured using a camera, a microscope, an inverted microscope, a wide-field fluorescent microscope, a scanning confocal microscope, a nonlinear optical microscope, a two-photon fluorescent microscope, and a coherent Raman microscope. Exemplary image-capturing devices additionally include, a high-resolution microscope, a TIRF microscope, a lattice light-sheet microscope, a super-resolution microscope, and a stochastic optical reconstruction microscope.

In one aspect of the present disclosure, a transcribed gene is selected from the group consisting of a chromosomal-derived gene and a plasmid-derived gene. Of course, it will be appreciated by one of skill in the art that the methods are not limited to obtaining sequence information from a single transcribed gene, rather, the methods provide whole-genome (or whole transcriptome) sequencing.

### Microfluidic Devices

The present disclosure provides microfluidic devices which find use, for example, in the disclosed methods and systems. In some embodiments, a microfluidic device according to the present disclosure comprises at least one lane, wherein each lane comprises an inlet, an outlet, and a plurality of separate chambers. In various embodiments, the microfluidic device comprises, 1, 2, 3, 4, 5, 6, 7, 8, 9, 10, 15, 20, 25, 30, 35, 40, 45, 50, 55, 60, 65, 70, 75, 80, 85, 90, 95 or 100 lanes or more. In this way, single cells are captured and imaged in serial, and then they are all processed in parallel.

In one embodiment, an apparatus (e.g., a “microfluidic device”) is provided comprising a fluidics cartridge (e.g., chip or micro-chip) comprising at least one lane including an inlet, an outlet, and a plurality of separate chambers, the inlet adapted to receive a collection of cells. In some embodiments, the apparatus further comprises a system comprising a cartridge receptacle adapted to receive the fluidics cartridge; a pump adapted to be in fluid communication with a reagent container containing a reagent and the inlet of the fluidics cartridge, the pump being configured to flow the reagent from the reagent container into the inlet of the fluidics cartridge to cause a single cell from the collection of cells to be isolated within one of the plurality of chambers of the fluidics cartridge; and an imaging assembly adapted to obtain image data of the single cell isolated within the one of the plurality of chambers of the fluidics cartridge.

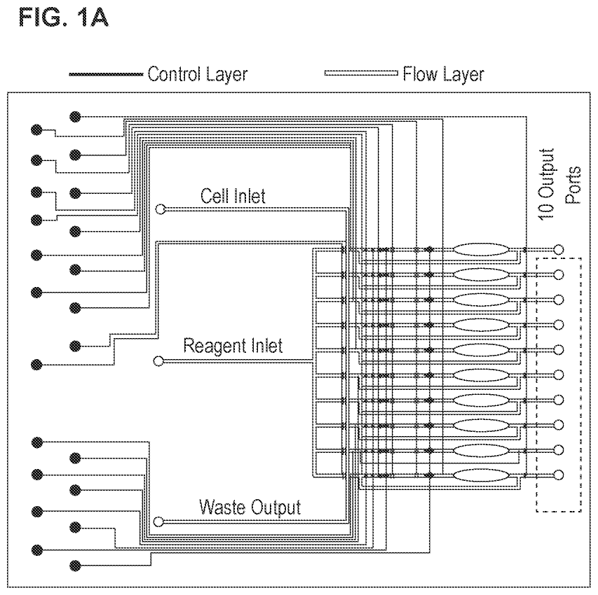

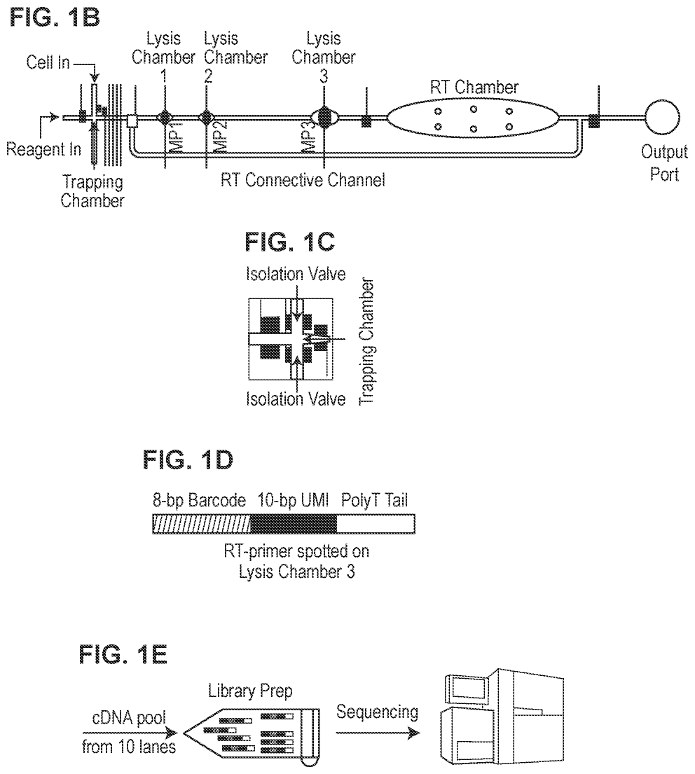

In some embodiments, the at least one lane optionally comprises separate chambers that allow (a) the injection of a collection of cells, (b) trapping of a single cell (e.g., a trapping chamber), (c) holding of the single cell, (d) lysis of the single cell (e.g., one or more lysis chambers), (e) digestion of the single cell, (f) ligation of primers to nucleic acid from the lysed single cell (e.g., a reverse-transcription chamber that has been preloaded or imprinted with barcoded RT primers), (g) amplification the nucleic acid (See, e.g., FIGS. 1A-1E). As described herein, the one or more lane further comprises inlets, outlets, and/or valves dispersed between one or more or all of the chambers. As described herein, the image data capture occurs, in some embodiments, while a single cell is in a cell trapping chamber. As described herein, each one or more lane further comprises in some embodiments an inlet to allow the injection of a reagent.

In some embodiments, a microfluidic device described herein further comprises a processor configured to access and process the image data to determine a cellular location of the DNA within the single cell.

In some embodiments, a microfluidic device described herein further comprises one or more valves adapted to constrain the single cell within the one of the plurality of chambers. In some embodiments, the valves are actuatable to flow the single cell from one chamber to another one of the plurality of chambers.

In some embodiments, a microfluidic device described herein further comprises a waste line coupled to the one of the plurality of chambers and adapted to selectively flow cellular debris to a waste reservoir.

In various embodiments, the isolation of a single cell, imaging of the single cell, and DNA amplification occurs in less than 1, 2, 3, 4, 5, 6, 7, 8, 9, 10, 30, 60, or 90 minutes.

Thus, in one embodiment, the present disclosure provides an apparatus, comprising: a fluidics cartridge comprising at least one lane including an inlet, an outlet, a trapping chamber, and a lysis chamber containing a preloaded barcoded reverse-transcription primer, the inlet adapted to receive a collection of cells; a system comprising: a cartridge receptacle adapted to receive the fluidics cartridge; and a pump adapted to be in fluid communication with a reagent container containing reagent and the inlet of the fluidics cartridge, the pump being configured cause a single cell from the collection of cells to be isolated within the trapping chamber and being further configured to flow the reagent from the reagent container into the inlet of the fluidics cartridge to cause the single cell within the trapping chamber to flow to the lysis chamber. In one aspect, the apparatus further comprises an imaging assembly adapted to capture image data of the single cell within the trapping chamber.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

The following materials and methods were used in in the Examples described herein.

### HEK293T Cell Culture and Single-Cell Suspension Preparation

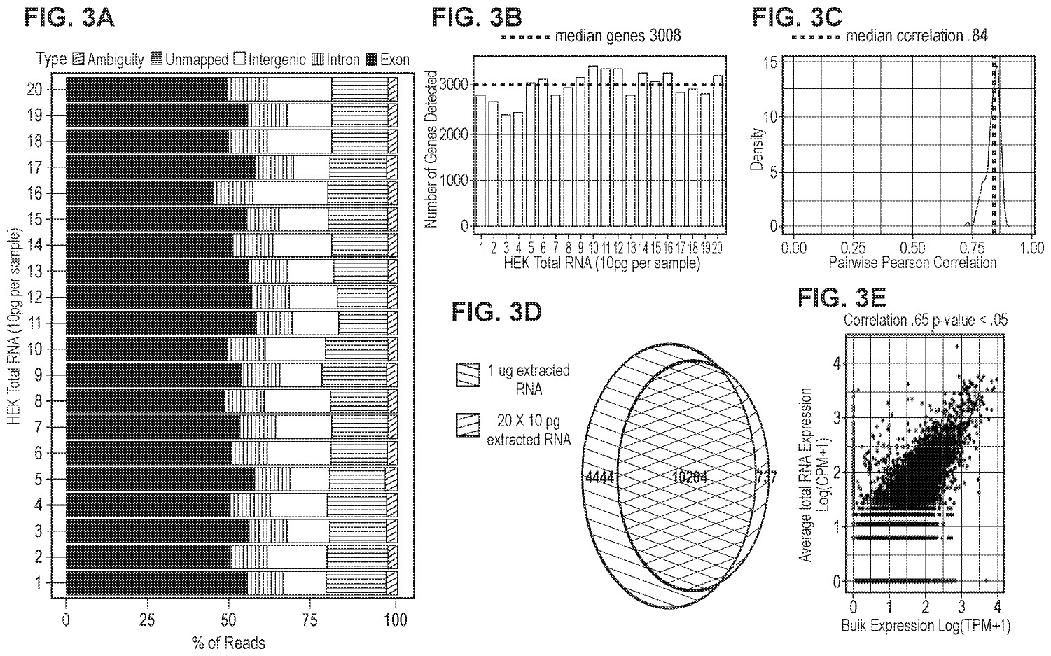

HEK293T cells were obtained from the UCSF cell repository, and cultured in DMEM medium (Gibco, 10566-016) supplemented with 10% vol/vol FBS and containing 1% vol/vol Penicillin-Streptomycin (Gibco). The cell culture was maintained at 37° C. in a humidified incubator containing 5% vol/vol CO₂. Confluent cells were passaged using TrypLE (Gibco, 12563011) with a 1:25 split in a new T25 flask (Falcon, 353109). For generating HEK293T single-cell suspensions for μCB-seq vs mcSCRB-seq comparisons (FIG. 3), cells were first grown to 100% confluency. The cells were then resuspended in 1 mL TrypLE and 5 mL of growth media and centrifuged at 1,200 rpm for 4 min. After centrifugation, the supernatant was removed and the cell pellet was washed with 1 mL of PBS (Corning, 21-040-CV). The cells were centrifuged again and this process was repeated for a total of three PBS washes to remove cell debris. Finally, the concentration of the cell suspension was adjusted in ice-cold PBS to 700 cells/μL using a hemocytometer (Hausser Scientific). After this, the cell suspension was always stored on ice throughout the course of device operation. In most experiments, around 50 μL of the single-cell suspension was aspirated into a gel-loading pipette tip and placed into the device, although the full volume was rarely completely used, and it is possible to decrease this volume in situations where the sample is limited.

### Preadipocytes Cell Culture

Human preadipocytes were provided by our collaborators in the Tseng lab at Joslin Diabetes Center. The cells were isolated from the deep neck region of a deidentified individual using the protocol in Xue et al. and immortalized to allow for cell culture and expansion. For culturing, Preadipocytes were grown in DMEM medium (Corning, 10-017-CV) supplemented with 10% vol/vol FBS and containing 1% vol/vol Penicillin-Streptomycin (Gibco). The cell culture was maintained at 37° C. in a humidified incubator containing 5% vol/vol CO₂. 80% confluent cells were passaged using 0.25% trypsin with 0.1% EDTA (Gibco; 25200-056) for a 1:3 split in a new 100 mm cell culture dish (Corning).

### Single-Cell Membrane Staining Protocol

HEK293T cells and Preadipocytes were stained with CellBrite™ Green (#30021) and Red (#30023) Cytoplasmic Membrane Labeling Kits respectively using manufacturer's protocol. Briefly, cells were suspended at a density of 1,000,000 cells/mL in their respective normal growth medium. 5 μL or 10 μL of the Cell Labeling Solution was then added per 1 mL of cell suspension for HEKs and Preadipocytes respectively. Cells were then incubated for 20 minutes (HEKs) or 40-60 minutes (Preadipocytes) in a humidified incubator containing 5% vol/vol CO₂. Cells were then pelleted by centrifugation at 1,200 rpm for 4 min. After centrifugation, the supernatant was removed and cells were washed in warm (37° C.) medium. Cells were centrifuged again and the process was repeated for a total of 3 growth medium washes for HEKs and 1-3 growth medium washes for Preadipocytes. Cells were then centrifuged a final time at 1,200 rpm for 4 minutes and resuspended in ice-cold PBS(Corning, 21-040-CV) to a concentration of 700 cells/μL adjusted using a hemocytometer (Hausser Scientific). The cells were then stored on ice throughout the μCB-seq device operation.

### Bulk RNA-sequencing and Analysis

RNA was extracted from HEK293T cells using the RNeasy Mini Kit from Qiagen (74104) with the QlAshredder (79654) for homogenization. RNA library preparation was performed with lug of total RNA input quantified by Qubit fluorometer using the NEBNext Poly(A) mRNA Magnetic Isolation Module (E7335S) followed by NEBNext Ultra II RNA Library Prep Kit for Illumina (E7770S). Paired-end 2×150 bp sequencing for RNA-seq library was performed on the Illumina Novaseq platform for a coverage of approximately 63 million read pairs. Adapters were trimmed using trimmomatic (v0.36; Bolger et al. 2014; ILLUMINACLIP:adapters-PE.fa:2:30:10 LEADING:3 TRAILING:3 SLIDINGWINDOW:4:15 MINLEN:36, where adapters-PE.fa is:

>PrefixPE/1
(SEQ ID NO: 1)
TACACTCTTTCCCTACACGACGCTCTTCCGATCT
>PrefixPE/2
(SEQ ID NO: 2)
GTGACTGGAGTTCAGACGTGTGCTCTTCCGATCT)

Reads were aligned to the CellRanger GRCh38 index (reference) using STAR. Paired-reads aligning to the exonic regions were quantified using the featurecounts command in the Subread package. Chimeric reads and primary hits of multi-mapping reads were also counted towards gene expression levels. The filtered CellRanger GRCh38 gene annotation file was used as the input for transcript quantification. The fragment counts matrix so obtained was converted to Transcripts Per Kilobase Million mapped reads (TPM) using the lengths for each gene as calculated by the featurecounts command in the Subread package. Genes with TPM>0 were defined to be reliably detected in bulk RNA-sequencing measurement.

### Confocal Imaging

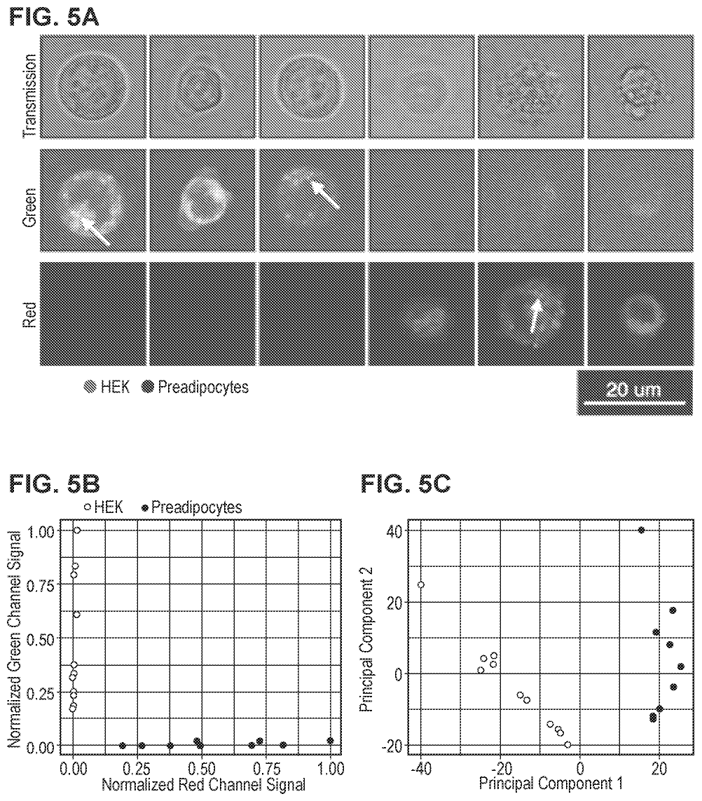

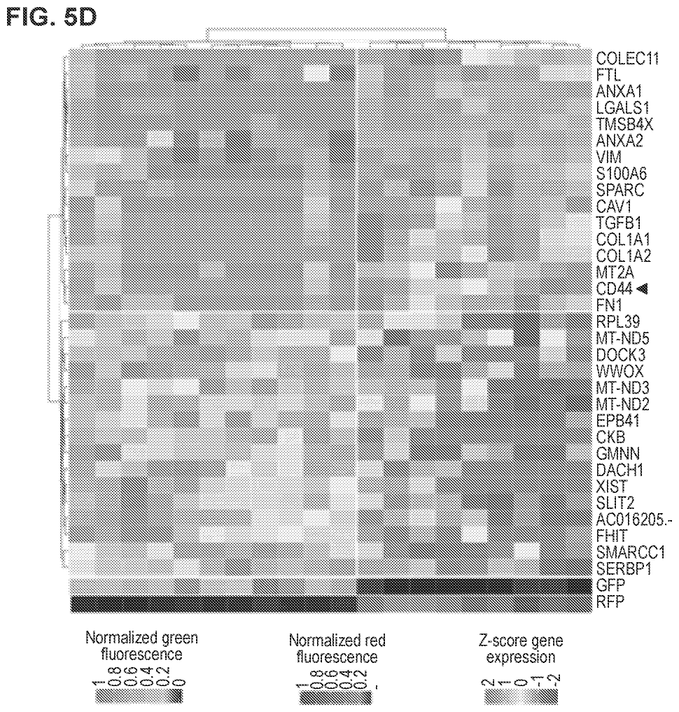

Fluorescence confocal imaging of cells was performed in the trapping chamber of the μCB-seq device using an inverted scanning confocal microscope (Leica, Germany), and with a 63× 0.7 NA long-working-distance air objective. As outlined before, HEKs were stained using CellBrite™ Green dye and Preadipocytes were stained using CellBrite™ Red dye. Each cell was excited by two continuous-wave lasers, a 488 nm Ar/Kr laser and a 633 nm He/Ne laser, for concurrent imaging in the green and red channels respectively. Bandpass filters captured backscattered light from 490-590 nm at the photomultiplier tube in the green channel (Green-PMT), and from 660-732 nm at the photomultiplier tube in the red channel (Red-PMT), with the pinhole set to 1 Airy unit. A third PMT simultaneously captured a scanning transmission image using the unfiltered forward-scattered light. The imaging resolution was Rayleigh-limited, with a scanning zoom of 2.2× to achieve a Nyquist sampling rate of 207 nm per pixel (as calculated for the Ar/Kr laser with a smaller wavelength). Each image was 8-bit, grayscale and 512×512 pixels in size. Since individual HEK cells and Preadipocytes internalized varying amounts of membrane stain, the PMT gain which utilized the entire range of bit-depth (0-255) differed from one cell to another. Therefore, stained HEK and Preadipocyte cell suspensions were first imaged on a #1.5 coverslip for adjusting the range of Green-PMT gain (range: 524.6) and Red-PMT gain (range: 512-582). We measured a maximum gain of 524.6 in the green channel and 582 in the red channel to observe cellular features, and therefore set the background PMT gain to an even higher value of 600, to validate that lack of features in background images was not because of low PMT gain. In all our images, the focal plane was positioned at the cross-section with maximum fluorescence intensity. The final images were Kalman-integrated over 6 frames to remove noise. Images in FIG. 5A have been adjusted to highlight cellular features. However, no adjustment was done for quantitative image processing.

### Quantitative Image Processing

To quantify the fluorescence signal intensity in individual HEKs and Preadipocytes labeled using the CellBrite™ Green and Red dye respectively, a custom image analysis script was written in Python (v3.7.1) using the skimage package (v0.20.2) and multi-dimensional image processing (ndimage) package from the SciPy (v1.2.1) ecosystem. As described herein, each cell had two fluorescence images, one green-channel confocal image, and one red-channel confocal image. Depending on the cell-type, one of the channels exhibited cellular signal (green for HEK and red for Preadipocytes) and the second channel conversely was a control image. For images of individual HEK cells and Preadipocytes, all green-channel and red-channel images respectively were analyzed to generate a cell mask (as described herein). The pixels constituting the cell mask were designated as foreground pixels and the remaining pixels were designated as background pixels. The fluorescence signal to noise ratio (SNR) was then quantified as the ratio of mean foreground pixel intensity over mean background pixel intensity. The same pixel annotation (for foreground and background pixels) was also used in the control images to quantify SNR in the second channel. In essence, the SNR was quantified in both green and red channels for each cell and these values were normalized to linearly scale between 0 and 1 for FIG. 5B and FIG. 5C. For cell mask generation, grayscale images were first gaussian filtered to remove noise using the ndimage.gaussian_filter command with sigma set as 1. The filtered images were converted into binary images using Otsu Thresholding from the skimage package. Pixels with value 1 in the binarized images were annotated as foreground and pixels with value 0 were annotated as background.

### Principal Component Analysis, Clustering and Differential Gene Expression Analysis

For membrane-stained HEKs and Preadipocytes, principal component analysis (PCA), clustering, and differential gene expression analysis were performed using the Seurat package (v3.1.1) on R programming language (v3.5.2). First, the umi-count matrix generated using zUMIs at a read depth of 125,000 per cell was read using the readRDS command. The count matrix was then used to create a Seurat object with no filtering for either cells or genes. The umi-count matrix was log-normalized with a scaling factor of 10,000 using the NormalizeData command. The top 2,000 most variable genes in the full dataset were identified using the variance-stabilizing transformation (vst) method implemented by the FindVariableFeatures command. The normalized count matrix was then scaled and centered to generate the Z-scored matrix using the ScaleData command. The first and second principal components were then calculated based on the Z-scored expression values of the 2,000 variable genes using the RunPCA command and the reduced space visualization was plotted using the ggplot2 package (v3.1.0) in R.

For clustering using Seurat, first, a K-Nearest Neighbor graph (KNN) was constructed using the cell embeddings in the PCA space (K=5). The generated KNN graph was then used to construct a Shared Nearest Neighbor (SNN) graph by calculating the Jaccard index between every cell and its nearest neighbors using the FindNeighbors command. Using the SNN graph, the clusters were then identified using the FindClusters command with the resolution parameter set to 0.1. At this resolution, HEKs and Preadipocytes separated into two clusters as visualized in the PCA space (FIG. 5C). After clustering, differentially expressed genes (logFC>0.5 and adjusted p-values<.05) between the two clusters were identified by fitting a negative binomial generalized linear model (negbinom test) on the raw umi-count matrix as implemented in the FindAllMarkers command. Z-scored expression values of the top 16 upregulated genes for each cell-type were then color mapped in a Heatmap plot using the ComplexHeatmap package. ComplexHeatmap was also used to perform unsupervised hierarchical clustering of single cells and genes using the euclidean distance metric and complete linkage classification method. Imaging heatmaps, with normalized green- and red-channel SNR as the data points, were also plotted using the ComplexHeatmap package.

### Control and Flow Mold Fabrication

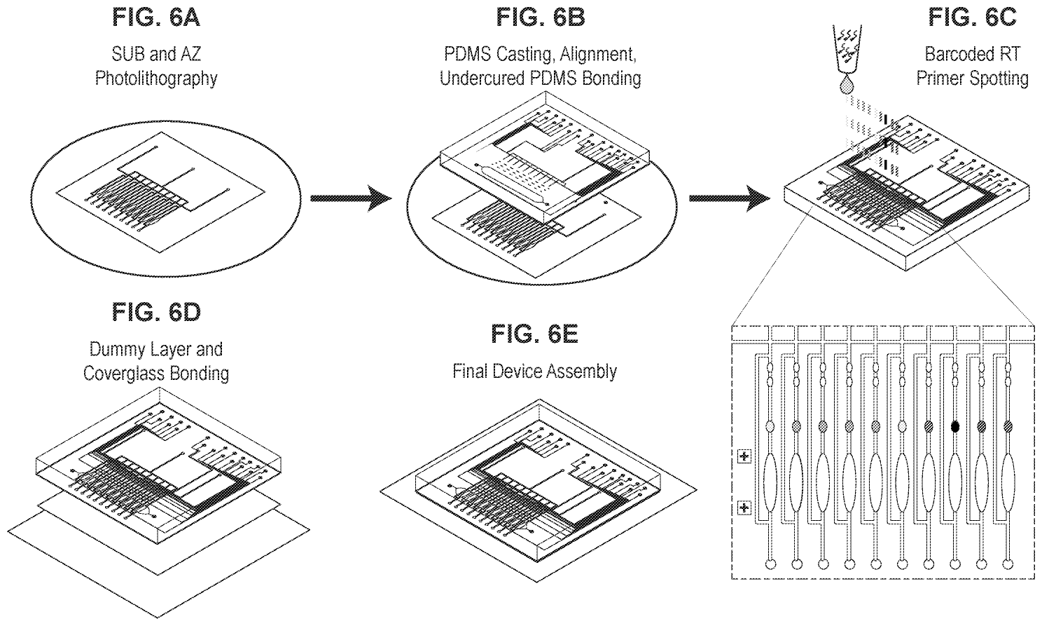

Two molds, a control mold and a flow mold, were patterned on silicon wafers (University Wafers, #S4P01SP) with photolithography (FIG. 6A). Patterns for the control and flow molds were designed in AutoCAD (Autodesk) and printed onto 25,400 dpi photomasks (CAD/Art Services, Inc., Bandon, Oreg.). The silicon wafers were first thoroughly cleaned using acetone, isopropyl alcohol, and water. The wafers were then baked at 150° C. for 10 min to dehydrate the surface. For the control mold, a 5 μm dummy layer of SU8-2005 (MicroChem) was first spin-coated at 3,000 rpm for 30 sec. The resist-coated mold was then baked at 65° C. for 1 min and 95° C. for 2 min and exposed to UV radiation with no mask for 10 sec. After exposure, the mold was again baked at 65° C. for 1 min and at 95° C. for 3 min and allowed to cool to room temperature. After dummy layer deposition, a dollop of SU8-2025 negative photoresist (MicroChem) was poured onto the control mold directly and then spun at 3,000 rpm for 30 sec, yielding a 25-μm layer. Then, the wafer was baked on a hotplate at 65° C. for 1 min and then at 95° C. for 5 min. The resist-coated wafer was exposed to a 150 mJ/cm² dose of UV radiation through a negative mask (clear features and opaque background) imprinted with the control circuit using a photolithography aligner. After exposure, the wafer was again baked at 65° C. for 1 min and 95° C. for 5 min. The wafer was then submerged in SU-8 developer and gently agitated until the unexposed photoresist was removed, leaving the positive control features. Then, the wafer was carefully washed with isopropyl alcohol and blow-dried. The mold was baked at 150° C. for at least 20 min before further use.

The flow mold was fabricated using two photoresists to achieve multiple-height features. The flow channels were fabricated using the positive photoresist AZ 40XT-11D (Integrated Micro Materials, Argyle, Tex.) and the taller reaction chambers were fabricated using the negative SU8-2025 photoresist. The flow mold was first spin-coated with a 5 μm dummy layer of SU8-2005 and processed the same as described for the control mold above. After dummy layer deposition, a dollop of AZ 40XT-11D positive photoresist was poured onto the flow wafer directly and then spun at 3,000 rpm for 30 s, yielding a 20-μm layer. After baking at 65° C. for 1 min and 125° C. for 6 min, the photoresist was then exposed to a 420 mJ/cm² dose of UV light through a high-resolution positive mask containing the flow circuit design and developed in AZ400K developer. The mold was then baked again at 65° C. for 1 min and at 105° C. for 100 sec to reflow the positive resist and create rounded channels. Negative photoresist (SU8-2025) was then used for building the reaction chambers using the same protocol as described for the control mold above.

### PDMS Device Fabrication

Multilayer PDMS devices were bonded together by on-ratio (10:1) bonding of RTV-615 (GE Advanced Materials) (Lai et al.). The control and flow molds were exposed to chlorotrimethylsilane (Sigma-Aldrich) vapor for 30 minutes before soft lithography to facilitate PDMS releasing from the mold. After mixing and degassing of PDMS, 50 g of PDMS was cast onto each control mold and baked at 80° C. for 15 min to partially cure the PDMS slabs (FIG. 6B). Control ports were punched and flow molds were spin-coated with a PDMS layer at a speed of 2,000 rpm for 60 sec. Flow layers were partially cured at 80° C. for 5 min, after which control slabs were aligned and placed atop flow PDMS(FIG. 6B). PDMS assemblies were cured at 80° C. for a further 10 min, after which devices were peeled off of the Si wafer. Flow ports were punched and assemblies were placed upside-down in preparation for primer spotting. In a clean hood, 0.2 μL of 1.5 μM barcoded μCB-seq primer was manually spotted in lysis chambers using a P2 pipette, with each lane receiving a unique, known barcode sequence (FIG. 6C). Primers were allowed to dry while a PDMS dummy layer was spin-coated and partially cured on a blank, silanized Si wafer. Control+flow-layer PDMS assemblies were then placed onto the PDMS dummy layer for a 1.5 hr hard bake at 80° C(FIG. 6D). Final devices were bonded to #1.5 glass coverslips by O₂ plasma (Instrument) and placed at 4° C. for storage(FIG. 6E).

### Microfluidic Device Operation

Microfluidic devices were attached to an Arduino-based pneumatic controller (KATARA) in preparation for running on-chip library prep. Prior to single-cell experiments, the cell trapping line was flushed with nuclease-free water (nfH₂O) and incubated with 0.2% (wt/wt) Pluronic F-127 for 1 hr, leaving downstream chambers containing barcoded primers empty. Confluent cells were trypsinized, suspended at a concentration of 105 cells/mL in PBS, and drawn into the cell trapping line by peristaltic pumping action of the integrated microfluidic valves. Triton Buffer was first prepared by combining 0.2 μL RNase Inhibitor and 3.8 μL 0.2% (v/v) Triton X-100. Lysis buffer was then prepared by mixing 1 μL 1:100 5× Phusion HF Buffer, 2.5 μL Triton Buffer, 0.7 μL nfH₂O, and 0.8 μL 1% (v/v) Tween 20 in a 0.2 mL PCR tube. Lysis buffer was aspirated into a gel-loading pipette tip, which was inserted into the reagent inlet and pressurized. The reagent tree was dead-end filled with lysis buffer, and the device was transferred to a confocal microscope (Leica) for cell trapping and imaging.

Cells were drawn along the cell input line by peristaltic pump and manually trapped in the trapping chamber for imaging, which was carried out by the protocol described in Confocal Imaging. After imaging, the chamber's individually-addressable valve was opened in concert with the reagent input valve, allowing lysis buffer to push the trapped cell into a lysis chamber containing dried, uniquely barcoded RT primers. After all cells were trapped, primers were resuspended by pumping action of the microfluidic paddle above the lysis chamber. The microfluidic device was transferred to a thermal block for cell lysis at 72° C. for 1 min, after which the block was cooled to 4° C. During cooling, the reagent inlet was flushed with 20 μL nuclease-free water and dried with air. Reverse transcription mix was then prepared in a 0.2 mL tube by mixing 0.8 μL 25 mM each dNTP mix, 4 μL 5× Maxima H-Buffer, 0.4 μL 100 μM E5V6 TSO, 5 μL 30% PEG 8000, 6.4 μL nfH₂O, 0.2 μL 1% Tween 20, and 0.2 μL 200 U/μL Maxima H-Reverse Transcriptase. Reverse transcription mix was injected into the reagent inlet to dead-end fill the reagent tree. Potential crosstalk was minimized by closure of the trapping valve to isolate all cell lanes after the reagent inlet wash. The RT ring, and individual valves were then opened to allow RT mix to dead-end fill all lanes. Reverse-transcription was carried out for 90 min at 42° C., with the ring peristaltic pump operating at 1 Hz to accelerate diffusive mixing of cell lysate, reverse transcription mix, and barcoded primers. Following reverse transcription, the chip was cooled to 4° C. and the reagent inlet was washed and dead-end filled with nuclease-free water. Barcoded cDNA was eluted in a volume of 1.7 μt per lane into gel loading pipette tips and pooled in a single PCR tube for downstream single-pot reactions.

Exonuclease digestion was carried out on the 17 μL of pooled library by adding 2 μL Exonuclease Buffer (10×) and 1 μL 20 U/μL Exol, with no concentration steps required, followed by incubation at 37° C. for 20 min, 80° C. for 10 min, and cooling to 4° C. Following eonuclease digestion, the following reagents were added to the library tube for PCR: 1.5 μL 1.25 U/pL Terra Direct Polymerase, 37.5 μL 2× Terra Direct Buffer, 1.5 μL 10 μM SINGV6 Primer, and 14.5 μL nfH₂O. PCR was carried out with the following protocol: 3 min at 98° C. followed by 17 cycles of (15 sec at 98° C., 30 sec at 65° C., 4 min at 68° C.), followed by 10 min at 72° C. and a 4° C. hold. Post-PCR libraries were size-selected with AmPure XP beads using a 0.6:1 Beads:Library volume ratio. Final libraries were run through the Nextera XT tagmentation protocol, with the PNEXTPT5 custom primer (Supplementary Table 2) substituted for the P5 index primer as in mcSCRB-seq. Indexed libraries were pooled and sequenced on an Illumina MiniSeq.

### mcSCRB-seq In-Tube Library Preparation

For mcSCRB-seq in-tube experiments, 96-well plates were first prepared with 10 barcoded primers and lysis buffer according to the mcSCRB-seq protocol, with the only difference being the use of μCB-seq RT primers instead of standard mcSCRB-seq ones. For total RNA experiments, 1 μL of 10 pg/μL Total RNA was directly pipetted into each well. For single-cell experiments, the CellenONE X1 instrument was used to individually deliver a single HEK cell into each well. Following cell delivery, the mcSCRB-seq protocol was followed directly, but with a 1:1 ratio of AmPure XP beads to pool all cDNA after RT as opposed to the manual bead formulation from standard mcSCRB-seq.

### Single-Cell and Total RNA Sequencing Data Processing

Filtering, demultiplexing, alignment, and UMI/gene counting were carried out on the zUMIs pipeline for all samples, using the GRCh38 index for STAR alignment. The gtf file that is recommended for the 10× CellRanger pipeline for standardization of gene counts was provided. Reads with any barcode or UMI bases under the quality threshold of 20 were filtered out, and μCB-seq barcode sequences were supplied in an external text file. UMIs within 1 hamming distance were collapsed to ensure that molecules were not double-counted due to PCR or sequencing errors. For this analysis, cell barcodes were not collapsed based on their hamming codes. Yam1 files for analysis of each dataset are provided in the supplement. For the Total RNA μCB-seq dataset (TC012), the quality of the 3rd base of Read 1 was poor due to the fact that all barcodes in the sequencing run had an Adenine at that position. Therefore, fastq files for this dataset were edited to remove the third base, and truncated barcode sequences were provided to zUMIs to match. This modification did not affect the information content or quality of the processed library.

Downstream data tidying and analysis was carried out in a Jupyter notebook with an R kernel, which can be found in the supplement. A Packrat library snapshot is also provided that contains all necessary packages for this analysis.

### Total RNA Chamber Volume Measurement

When measuring chamber volume for Total RNA experiments in the μCB-seq device, we initially observed a difference in height between the μCB-seq flow molds and the channels of the finalized PDMS μCB-seq devices. Flow molds were measured by Dektak profilometer, giving an imaging chamber height of 29 μm. When imaging the corresponding chamber on the μCB-seq device via Coherent anti-Stokes Raman spectroscopy (CARS), a chamber height of 53.5 μm was recorded. Profilometry was not feasible for the closed μCB-seq device, so the CARS measurement was used at the risk of overestimating volume and loading less than 10 pg Total RNA into the μCB-seq device. To measure chamber volume, the isolation valves were pressurized on a μCB-seq device and acquired a z-stack of the resultant air-filled imaging chamber. Images were thresholded in ImageJ and manually outlined to record the cross-sectional area of each imaging chamber slice. The volume of the chamber was estimated by a Riemann sum to ensure that chamber volume erred on the larger side. The chamber volume measured by this method was 1.88 nL, which resulted in our conservative input concentration of 5.31 ng/μL Total RNA to ensure no more than 10 pg of RNA was processed in each lane of the μCB-seq device for direct comparison against mcSCRB-seq in-tube.

### Example 1

### Microfluidic Device Design and μCB-seq Workflow

μCB-seq is implemented, in one aspect of the disclosure, on a PDMS-based microfluidic device with two functional layers, an upper control layer, and a bottom flow layer (FIG. 1A). Each orthogonal intersection of control and flow channels on the device forms an integrated microfluidic valve, which can be actuated to control fluid flow by pressurizing the control channel. Pressure in control channels is regulated by solenoid valves operated via a programmable computer interface (J. A. White and A. M. Streets, HardwareX, 2017, 3, 135-145). Each reaction lane has a modular design to allow for imaging and multistep library preparation including cell lysis and reverse-transcription (RT). The imaging module consists of an imaging chamber flanked by two isolation valves, the lysis module consists of three reaction chambers (used as one large chamber in this demonstration) and the RT module consists of a larger reaction chamber with a connective channel forming a closed mixing ring with the reaction lane (FIG. 1B and 1C). During chip operation, a suspension of single cells is loaded into the cell inlet and directed towards the imaging module using pressure-driven flow. Once a cell reaches an imaging chamber, the isolation valves are pressurized to immobilize the cell for imaging (FIG. 1C). If multiple or apoptotic cells are trapped, the isolation valves are reopened, and the unwanted cell is discarded to the waste output. After imaging, the selected cell is then ejected from the imaging chamber into the lysis module of its reaction lane using a pressure-driven flow of the lysis mix from the reagent inlet. Once all 10 lysis regions are filled with cell lysates, processing proceeds in parallel for all 10 cells.

During chip fabrication, RT primers with known barcode sequences are spotted in the 3^{rd} reaction chamber of the lysis module for each reaction lane. By this method, each reaction lane is indexed by two pieces of information: (1) a known barcode sequence and (2) its spatial location on the device. Since microscopy occurs upstream of library preparation in the same reaction lane, the acquired image can be annotated by the same address as the reaction lane. As a result, all sequencing reads with the same known barcode sequence can be linked to cell images with the corresponding spatial address. Barcode sequences used in this way are a subset of 8-nt long Hamming-correctable barcodes (Bystrykh LV (2012), PLoS ONE 7(5)) selected for 50% GC content and minimal sequence redundancy. The unique molecular identifier (UMI) sequence in the RT primers is 10-nt long (FIG. 1D).

Positioned above the three reaction chambers in the lysis module are mixing paddles, which are used to accelerate homogenization (FIG. 1B). To re-suspend the barcoded RT primers in cell lysate, the mixing paddle on top of the 3^{rd} reaction chamber is actuated. The entire chip is then placed on a temperature-controlled platform for incubation at 72° C. for 1 minute of lysis and cooled to hybridize the RT primers with mRNA transcripts. The reagent input line is then flushed and filled with RT mix, which is injected to dead-end fill the ring-shaped RT module of each reaction lane. Reverse transcription is carried out for 1.5 hours at 42° C., during which the mixing paddles are actuated in a peristaltic manner to circulate the relatively viscous RT mix throughout the closed-loop of each reaction lane.

The total reaction volume of all preparation steps per lane is 227 nL, which is a 44-fold decrease from the in-tube mcSCRB-seq protocol (10 μL). After RT, all lanes are independently flushed with 1.7 μL of nuclease-free water to recover cDNA, and pooled into a single tube using gel-loading pipette tips for a total volume of 17 μL. Additional exonuclease digestion and cDNA amplification followed by purification and Nextera library preparation are performed in a single tube using the conventional mcSCRB-seq protocol. cDNA libraries representing whole single-cell transcriptomes are then sequenced on a next-generation sequencing platform (FIG. 1E).

### Example 2

### Microfluidic Device Fabrication Integrated with Addressable Barcode Spotting

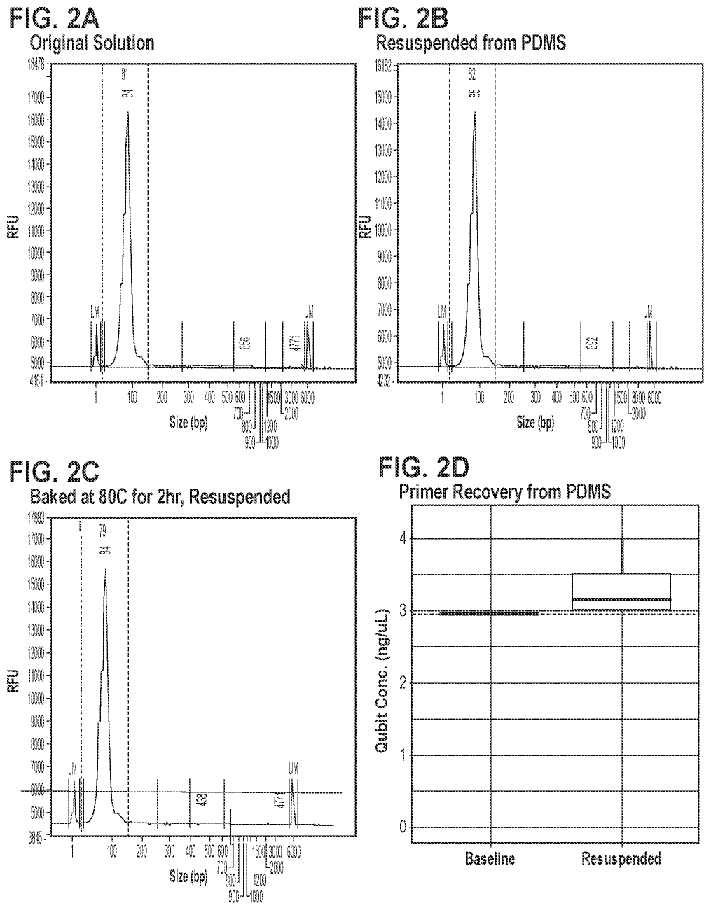

CB-seq is enabled by a novel fabrication method that combines multilayer soft lithography and DNA array printing to index reaction chambers on the device with known DNA barcodes (M. A. Unger, et al., Science (80-.)., 2000, 288, 113-116). Multilayer chip fabrication has long been used to create microfluidic devices with integrated valves and pumps which can be actuated for precise fluidic manipulation of cells, buffer exchange, and continuous-flow mixing of reagents. These capabilities enable implementation of multistep reactions for library preparation on such devices, but reagent carryover from the single inlet makes it challenging to run uniquely barcoded reactions without crosstalk. Our new fabrication method overcomes this, allowing us to preload the lysis module of μCB-seq devices with barcoded primers that are only re-suspended when contacted by aqueous cell lysate. This simple and robust method for integrating specific oligonucleotides within a PDMS device during soft lithography does not require any challenging alignment steps, since the reaction chamber itself serves both as fiducial and target for delivery of RT primers. To verify that RT primers can be successfully resuspended from PDMS after drying and baking, 2 uL droplets of 2 ng/uL μCB-seq primer were manually spotted on PDMS slabs, baked at 80° C. for 2 hr, and allowed to sit at room temperature for 24 hr. Primers were manually re-suspended in 2 uL of nuclease-free water and analyzed for concentration and fragment length. The μCB-seq primers show no noticeable degradation during the final baking at 80° C. and can be re-suspended with high efficiency (FIG. 2).

μCB-seq Device Fabrication. The μCB-seq device was designed in the push-down configuration with three layers: a thick upper control layer, a thin middle flow layer, and a thin lower dummy layer. An on-ratio PDMS-PDMS bonding technique was used as it avoids PDMS waste and provides a stable seal by partial crosslinking of a 10:1 base:crosslinker mixture with each new layer of the microfluidic device (A. Lai, et al., J. Micromechanics Microengineering,. DOI:10.1088/1361-6439/ab341e). The control and flow molds were patterned using standard photolithography techniques and exposed to chlorotrimethylsilane (Sigma-Aldrich) vapor for 30 minutes before soft lithography to facilitate PDMS releasing from the mold. PDMS mixture (RTV-615; GE Advanced Materials) was then spin-coated onto the flow mold and poured onto the control mold. The flow and control layers were partially cross-linked by baking for 6 and 15 min, respectively, at 80° C. The control layer slab was peeled from the mold, and holes were punched for control ports. The control layer slab was then aligned and placed atop the thin flow layer, after which the two-layer assembly was baked at 80° C. for 10 min. The assembly was peeled from the flow mold and fluidic inlet holes were punched.

The two-layer assembly was then inverted, exposing the open face of the device, and barcoded μCB-seq primers were spotted into the 3^{rd} reaction chamber in the lysis module of each reaction lane and allowed to dry. For this demonstration, a P2 micropipette was used to manually spot 0.2 uL of 1.5 uM μCB-seq primer in nuclease-free H₂O. While spotted barcodes dried, the bottom PDMS dummy layer was spun onto a blank, silanized silicon wafer and baked for 6 min at 80° C. The two-layer chip with dried barcodes was then carefully placed onto the dummy layer to close the device. The whole device was baked for 1.5 hr at 80° C. to complete the bonding. Finally, the assembled μCB-seq device was cut from the dummy wafer and bonded onto a #1.5 glass coverslip using oxygen plasma bonding.

### Example 3

### μCB-seq Yields High-Quality scRNA-Seq Libraries

μCB-seq library preparation can be considered a microfluidic implementation of the highly sensitive mcSCRB-seq protocol, which is a 3′ counting method using UMIs and cell barcodes to acquire a multiplexed absolute transcript count from each cell. The effectiveness of μCB-seq was evaluated by generating scRNA-seq libraries from 20 replicates of 10 pg total RNA isolated from HEK293T cells. Total RNA extracted from HEKs was diluted to a concentration of 5.31 ng/uL (10 pg per imaging chamber) and injected into the cell inlet. The 10 sets of isolation valves were then simultaneously actuated, and the contents of each imaging chamber were pushed into their respective reaction lanes for library preparation as described previously. The libraries were sequenced using the Illumina Miniseq platform with Read 1 encoding for the 8-nt μCB-seq barcode and 10-nt UMI, while Read 2 was used to sequence the cDNA fragment. After sequencing, all raw fastq files were analyzed using the zUMIs pipeline (S. Parekh, et al., Gigascience, 2018). In zUMIs, reads were filtered and mapped to the human reference genome (GrCh38) using STAR (A. Dobin, et al., Bioinformatics, DOI:10.1093/bioinformatics/bts635). Gene annotations were obtained from Ensembl (GRCh38.93) and filtered to remove biotypes such as pseudogenes. Quantification of aligned reads was done using the Subread package to generate expression profiles for each library (Y. Liao, et al., Nucleic Acids Res., DOI:10.1093/nar/gkt214). Throughout this study, genes detected were defined as those for which at least one UMI was detected with all bases having quality score >20.

The mapping statistics were first characterized for each of the 20 total RNA libraries. These metrics allowed us to evaluate the percentage of useful reads for downstream analysis. In all the replicates, a median of 53% of the reads mapped to exons, 11% to introns, 16% to intergenic regions, and 17% to no region in the human genome (FIG. 3A). These statistics are comparable to other 3′-barcoding-based sequencing protocols with a range of 29-57% exonic reads, 2-15% intronic reads and 6-23% unmapped reads. (Ding, X. et al., bioRxiv, 2019, 632216). Detection of reads from unspliced transcripts makes μCB-seq dataset compatible with vector-based single-cell analyses such as RNA Velocity (G. La Manno, et al., Nature, 2018, 560, 494-498). For this analysis however, only reads mapping to the exonic regions of the genome were quantified to generate a UMI count expression matrix. 10 pg total RNA sequencing libraries were information-rich with a median of 3008 unique genes detected at a shallow sequencing depth of 30,000 reads per sample (FIG. 3B). Since all total RNA replicates were prepared from the same bulk extracted RNA sample, any variation in gene expression between samples could be attributed to technical differences and stochastic sampling effects. Using μCB-seq, we observed a median pairwise Pearson coefficient of 0.84 (n=190 pairs) suggesting a strong similarity in gene expression within replicates processed across multiple reaction lanes and devices (FIG. 3C).

The performance of μCB-seq was evaluated by the overlap between genes detected in 10 pg total RNA measurements and bulk RNA-seq measurements using the NEBNext® Ultra™ II RNA Library Prep Kit. The final bulk library was prepared using 1000 ng of HEK total RNA and sequenced on the Illumina Novaseq platform. For comparison, we first pooled the transcriptomes of all 20 μCB-seq libraries of 10 pg total RNA for a total sequencing depth of 1.3 million reads and compared the genes detected with the genes mapped from 1.3 million bulk sample reads (TPM>0). With the same total number of reads, the 200 pg of μCB-seq libraries detected ˜70% of genes picked up by bulk RNA-seq of 1000 ng total RNA (FIG. 3D). There were over 700 genes that were detected in μCB-seq but not bulk mRNA-seq. These are likely a combination of low-abundance transcripts that were below the detection limit in ensemble measurements and transcripts that are not primed or reverse-transcribed due to molecular differences by protocol. On average, a 10 pg total RNA sample had a similar gene expression as the bulk measurement with a Pearson correlation of 0.65 (p-value<0.05, FIG. 3E). This shows that, an average 10 pg μCB-seq library forms a representative sample of the bulk transcriptome with a good correlation between expression levels.

### Example 4

### μCB-seq has Higher Sensitivity than In-Tube Protocol

In the context of whole-transcriptome sequencing, the sensitivity of a protocol can be understood as the percentage of RNA transcripts that are captured and converted into sequenceable DNA molecules in the final library. Multiplexed plate-based scRNA-seq protocols often rely on post-RT bead-based cleanup to pool and concentrate many single-cell cDNA libraries into a single tube for PCR. The cleanup is required to realize the ease-of-use benefits of early cell pooling, but bead purification necessarily incurs some sample loss during cDNA binding and elution. Since bead-based pooling occurs immediately after RT, the loss of molecules directly reduces the information content of the final library pool. This is in contrast to post-PCR bead cleanup, in which each molecule has many duplicates that contain the same information. The loss of unique cDNA molecules during bead-based pooling, therefore, translates to reduced sensitivity and gene detection capability for multiplexed scRNA-seq protocols. Microfluidic library preparation, on the other hand, allows for the pooling of hundreds of samples without the use of post-RT bead cleanup because each sample only occupies a nanoliter-scale volume on-chip. Moreover, using a microfluidic approach has been shown to increase the efficiency of mRNA capture during RT (A. M. Streets, et al., Proc. Natl. Acad. Sci., 2014, 111, 7048-7053). Since μCB-seq is a microfluidic implementation of the in-tube mcSCRB-seq protocol, it was hypothesized that μCB-seq will improve upon the high sensitivity of mcSCRB-seq. Only exonic reads were used for quantification for the following analyses since the conventional mcSCRB-seq protocol uses only exonic reads.

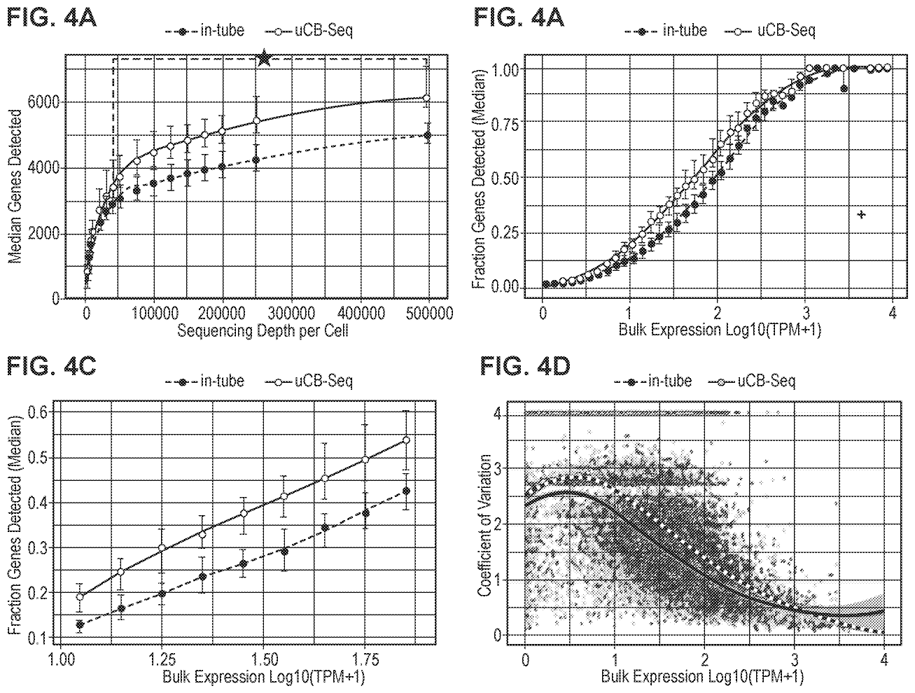

To practically compare the sensitivity of the two protocols, the number of genes detected using the μCB-seq and mcSCRB-esq protocols was benchmarked. scRNA-seq libraries were prepared from 18 HEK cells using μCB-seq and 16 HEK cells using mcSCRB-seq. All libraries were sequenced to an average depth of 500,000 reads per cell and downsampled to varying depths to assess the number of genes detected. The zUMIs pipeline was used to generate the count matrix for all sequencing depths. As expected, μCB-seq consistently detected more genes and UMIs, with significantly higher genes for depths >=40,000 reads per cell (p-value<0.01, two-group Mann-Whitney U-test, FIG. 4A). Moreover, μCB-seq libraries had a median of 21% intronic reads as compared to 15% in mcSCRB-seq which were not accounted for gene count quantification, making FIG. 4A a conservative estimate of the sensitivity improvements offered by the microfluidic protocol.

The sensitivity of μCB-seq and mcSCRB-seq was further evaluated by comparing the fraction of bulk genes that were detected in each single-cell protocol across the full range of expression levels. The bulk library was prepared from 1000 ng of HEK total RNA and sequenced to a saturating depth of ˜63 million reads, so it was assumed this bulk dataset is a relatively unbiased representation of the entire HEK transcriptome. Since μCB-seq detected more genes that mcSCRB-seq for the same sequencing depth, it was believed that these additional genes would increase the fraction of genes detected in the low-expression bins of the bulk dataset. All μCB-seq and mcSCRB-seq libraries were down-sampled to 200,000 reads per cell with 16 cells in each protocol. As anticipated, μCB-seq detected more genes than mcSCRB-seq across all expression levels with a substantial increase in the ability to detect low- and medium-abundance transcripts (FIG. 4B and 4D). Sensitivity was quantified as the bulk expression level (TPM) necessary for 50% detection probability and report that μCB-seq has ˜1.6 times higher sensitivity than mcSCRB-seq.

The scRNA-seq measurement precision was also assessed in the μCB-seq protocol as compared to mcSCRB-seq. Variation in gene count measurements between single-cell cDNA library preparations is caused by technical variation such as pipetting and human handling errors, sampling statistics, and true biological variation between cells. With microfluidics, it is possible to minimize the technical noise by automating and parallelizing library preparation reactions in lithographically defined volumes. As the noise associated with technical artifacts goes down, statistical power to parse out real biological variation is gained. Therefore, the benefits gained by the improved sensitivity of μCB-seq are contingent upon having low levels of technical variation. To quantify this, the coefficient of variation (CV) was calculated for genes detected across bulk, μCB-seq and mcSCRB-seq libraries as a function of bulk expression. Significantly lower variation in μCB-seq compared to mcSCRB-seq across the entire range of bulk expression except for very highly abundant genes (TPM>=560, FIG. 4C) was observed. Therefore, μCB-seq offers extremely high gene detection sensitivity and precision by eliminating lossy post-RT bead-based cleanup and carrying out library preparation in lithographically defined nanoliter-scale volumes.

### Example 5

### μCB-seq Links High-Resolution Optical Images with the Transcriptome of the Same Single Cell

Preloading lysis chambers with known barcode sequences allows making both imaging and sequencing measurements on the same single cell. High-resolution confocal images were linked with the transcriptomes of two differentially-labeled cell types. Two cell lines—HEK293T and adipocyte precursor cells (preadipocytes) (R. Xue, et al., Nat. Med., 2015, 21, 760-768)—were stained with CellBrite green and red cytoplasmic membrane dyes respectively. The cells were then suspended and processed on three μCB-Seq devices, one with both HEKs (n=4) and preadipocytes (n=3), one with just HEKs (n=7), and a third with just preadipocytes (n=6). Fluorescence confocal imaging was performed while cells were isolated in the imaging chambers using 488 nm and 633 nm lasers and with a 63× magnification 0.7 NA air objective. The cells were then ejected into their respective reaction lanes for library preparation on-chip followed by pooled PCR. All 20 libraries were sequenced on the Illumina MiniSeq platform for a minimum sequencing depth of 125,000 reads per cell. In this analysis, both intronic and exonic reads were used for generating a count matrix to utilize the introns detected by μCB-seq. After sequencing, reads were demultiplexed based on their cell barcodes, which allowed us to assign each cDNA read to the image of the cell from which the molecule originated.

FIG. 5A displays representative scanning-transmission and scanning-confocal images of HEKs and preadipocytes in both green and red channels confirming differential labeling of the two cell types (FIG. 5A). Since one cell was imaged at a time, high-resolution microscopy was used by scanning at the confocal Nyquist sampling rate for a resolution of 209 nm per pixel, which enabled observing subcellular features. Using distinct stains allowed determination of the cell type of each captured cell prior to sequencing-based analysis. As expected, quantification of the fluorescence signal in the green and red channels completely separated the two cell-types along the major axes (FIG. 5B). Importantly, groups of HEKs and preadipocytes identified using image analysis also presented as two distinct cell populations upon unsupervised clustering in the principal component space (FIG. 5C). No technical artifacts associated with three different chips were observed in the reduced space. In this case, μCB-seq optical imaging serves as a ground truth measure for naïve clustering of transcriptomic data from the same cells.

The sequencing dataset was further analyzed to understand the transcriptomic variations in this heterogeneous group of 20 cells. Differential gene expression analysis revealed 103 genes with logFC>0.5 and adjusted p-value<0.05. Interestingly, preadipocytes had an enriched expression of CD44, a mesenchymal stem cell surface marker which has been suggested to be expressed in adipogenic cells (Y. H. Lee, et al., Cell Cycle, DOI:10.4161/cc.27647; and Y. H. Lee, et al., Am. J. Physiol.-Regul. Integr. Comp. Physiol., DOI:10.1152/ajpregu.00355.2015). Unsupervised hierarchical clustering was also performed on the expression levels of the top 16 upregulated genes in the two cell types. All twenty cells were sorted into two distinct groups that accurately reflected their known cell type. As expected, there were two general subsets of genes: genes that showed upregulated expression in HEKs, and genes that showed upregulated expression in preadipocytes. Differential gene expression statistics were also coupled with fluorescence signal to gain another dimension on which to stratify cells and to provide a one-to-one mapping of each imaging data point to its corresponding sequencing data point (FIG. 5D). Importantly, the color scheme derived in the imaging heat maps in FIG. 5D reflects the actual two-channel fluorescence intensity from live-cell microscopy. Therefore, μCB-seq adds a phenotypic dimension on top of transcriptomic profiling upon which to characterize cell types.

In summary, by using a microfluidic approach in μCB-seq for library preparation, post-RT bead-based cleanup has been eliminated, operational errors are minimized, and nanoliter-scale, reproducible reaction volumes has been achieved. The microfluidic approach disclosed herein offers improvements in gene detection sensitivity as demonstrated by sequencing 16 HEK cells with both μCB-seq and the conventional in-tube mcSCRB-seq protocol. As shown in the Examples, using μCB-seq, a large portion of the bulk transcriptome was constructed by sequencing 20 replicates of 10 pg total RNA to a total depth of ˜1.3 million reads. The integration of on-chip valves in the device allows one to select cells of interest, making the μCB-seq platform applicable for studies focusing on rare cell populations (Y. Chen, et al., Lab Chip, 2014, 14, 626-645). On-chip isolation valves prevent cellular motion due to fluid flow, thereby allowing the acquisition of even prolonged spectroscopic measurements (K. J. Kobayashi-Kirschvink, et al., Cell Syst., 2018, 7, 104-117.e4) on the device. In terms of scaling, the throughput of μCB-seq can be increased tenfold with the current barcode list by using a microfluidic multiplexing strategy with a minimal increase in the peripheral operating equipment (T. Thorsen, et al., Science (80-.), DOI:10.1126/science.1076996; and W. H. Grover, et al., Lab Chip, DOI:10.1039/b518362f). Thus, the μCB-seq platform is a powerful tool for investigations aiming to understand the association between a phenotype and the transcriptome, thereby gaining a high-resolution fingerprint for a particular cell population identified using higher-throughput scRNA-seq protocols.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A method of determining the sequence of one or more transcribed genes from a single cell, said method comprising the steps of:
(a) administering a collection of cells to one lane of a microfluidic device under conditions that allow a single cell from the collection of cells to enter a first chamber in the microfluidic device;
(b) capturing a single cell in a trapping chamber of the microfluidic device;
(c) flowing the singe cell to a lysis chamber pre-loaded with barcoded reverse-transcription primers;
(d) preparing a barcoded cDNA library from the single cell using the barcoded reverse-transcription primers under conditions that allow barcoded cDNA preparation;
(e) sequencing the barcoded cDNA;
wherein steps (b)-(d) are carried out in in the microfluidic device.

2. The method of claim 1, further comprising determining the abundance of the one or more transcribed genes.

3. The method of claim 1 wherein step (b) additionally comprises the step of collecting a non-invasive measurement of the single cell.

4. The method of claim 2 wherein the non-invasive measurement comprises an optical measurement.

5. The method of claim 4, wherein the optical measurement is selected from the group consisting of spectroscopy, light scattering imaging, and fluorescent lifetime imaging.

6. The method of claims 4 wherein the optical measurement comprises capturing an image of the single cell.

7. The method of claim 6 wherein the image of the cell is captured from a device selected from the group consisting of a camera, a microscope, an inverted microscope, a wide-field fluorescent microscope, a scanning confocal microscope, a nonlinear optical microscope, a two-photon fluorescent microscope, and a coherent Raman microscope.

8. The method of claim 3, wherein the method additionally comprises the step of linking the sequence obtained in step (e) with the image captured in step (b), thereby correlating expression of one or more transcribed genes to a single cell morphology or phenotype.

9. The method of any of claims 1-8 wherein the preparing of barcoded cDNA of step (d) comprises the steps of: (i) lysing the cell, (ii) re-suspending the barcoded primers, (iii) administering reagents and applying temperatures that allow cDNA preparation, and (iv) collecting the barcoded cDNA library.

10. The method of claim 9 wherein the lysing step comprises contacting the cell with a cell lysing agent selected from the group consisting of ionic and non-ionic detergents, Triton X-100, sodium dodecyl sulfate (SDS), NP-40, and ammonium chloride potassium.

11. The method of any of claims 1-10 wherein the microfluidic device comprises 1-100 separate lanes, each comprising at least one chamber.

12. The method of claim 11 wherein each lane comprises 1, 2, 3, 4, 5, 6, 7, 8, 9, 10, 11, or 12 separate chambers.

13. The method of claim 12 wherein the cDNA from 1, 2, 3, 4, 5, 6, 7, 8, 9, or 10 lanes of the microfluidic device are pooled prior to sequencing.

14. The method of any of claims 1-13 wherein the transcribed gene is selected from the group consisting of a chromosomal-derived gene and a plasmid-derived gene.

15. The method of any of claims 1-14 wherein the cell is a bacterial cell, a eukaryotic cell or prokaryotic cell.

16. The method of claim 16 wherein the cell is a mammalian cell.

17. The method of claim 17 wherein the cell is a human cell.

18. A method of determining the sequence of one or more transcribed genes from a single cell, said method comprising the steps of:
(a) administering a collection of cells to one lane of a microfluidic device under conditions that allow a single cell from the collection of cells to enter a first chamber in the microfluidic device;
(b) capturing a single cell in a trapping chamber and collecting an image of the single cell;
(c) flowing the singe cell to a lysis chamber pre-loaded with barcoded reverse-transcription primers;
(d) preparing a barcoded cDNA library from the single cell using the barcoded reverse-transcription primers under conditions that allow barcoded cDNA preparation;
(e) sequencing the barcoded cDNA;
wherein steps (b)-(d) are carried out in in the microfluidic device.
